(19) United States
(12) Patent Application Publication
(10) Pub. No.: US 2026/0188507 A1
(43) Pub. Date: Jul. 2, 2026

(54) PREDICTING TREATMENT OUTCOMES AND POST-OPERATIVE RISK FOR CARDIAC SURGERIES

(71) Applicant: Texas Tech University System, Lubbock, TX (US)

(72) Inventors: Nandini Nair, Lubbock, TX (US); Dongping Du, Lubbock, TX (US)

(73) Assignee: Texas Tech University System, Lubbock, TX (US)

(21) Appl. No.: 19/129,648

(22) PCT Filed: Nov. 14, 2023
(86) PCT No.: PCT/US2023/037226

§ 371 Date: May 13, 2025

Related U.S. Application Data
(60) Provisional application No. 63/425,182, filed on Nov. 14, 2022.

(51) Int. Cl. G16H 50/30; G16H 10/60; G16H 20/10; G16H 50/20

(52) U.S. Cl. CPC G16H 50/30; G16H 10/60; G16H 20/10; G16H 50/20

(57) ABSTRACT

Embodiments of the present disclosure pertain to computer-implemented methods of predicting an outcome of a cardiac procedure in a subject by (1) receiving a plurality of health-related data from the subject; (2) feeding the health-related data into a risk prediction model that integrates the health-related data and predicts one or more outcomes of the cardiac procedure; and (3) generating an output from the risk prediction model, where the output includes the predicted outcomes of the cardiac procedure. The methods of the present disclosure may also include a step of (4) recommending a treatment decision based on the predicted outcomes of the cardiac procedure. Additional embodiments of the present disclosure pertain to a computing device that is operable for predicting an outcome of a cardiac procedure in a subject.

### CROSS-REFERENCE TO RELATED APPLICATIONS

This application claims priority to U.S. Provisional Patent Application No. 63/425,182, filed on Nov. 14, 2022. The entirety of the aforementioned application is incorporated herein by reference.

### STATEMENT REGARDING FEDERALLY SPONSORED RESEARCH

This invention was made with government support under 1728338 awarded by the National Science Foundation. The government has certain rights in the invention.

### BACKGROUND

Current methods and systems for predicting outcomes of cardiac surgical procedures suffer from numerous limitations. Embodiments of the present disclosure aim to address the aforementioned limitations.

### SUMMARY

In some embodiments, the present disclosure pertains to a computer-implemented method of predicting an outcome of a cardiac procedure in a subject. In some embodiments, the methods of the present disclosure include: (1) receiving a plurality of health-related data from the subject; (2) feeding the health-related data into a risk prediction model that integrates the health-related data and predicts one or more outcomes of the cardiac procedure; and (3) generating an output from the risk prediction model, where the output includes the predicted outcomes of the cardiac procedure. In some embodiments, the methods of the present disclosure also include a step of (4) recommending a treatment decision based on the predicted outcomes of the cardiac procedure.

Additional embodiments of the present disclosure pertain to a computing device that is operable for predicting an outcome of a cardiac procedure in a subject. In some embodiments, the computing device includes: (1) programming instructions for receiving a plurality of health-related data from the subject; (2) programming instructions for feeding the health-related data into a risk prediction model that is operable to integrate the health-related data and predict one or more outcomes of the cardiac procedure; and (3) programming instructions for generating an output from the risk prediction model, where the output includes the predicted outcomes of the cardiac procedure. In some embodiments, the computing devices of the present disclosure also include (4) programming instructions for recommending a treatment decision based on the predicted outcomes of the cardiac procedure.

### DESCRIPTION OF THE DRAWINGS

FIGS. 1A and 1B provide illustrations of a method (FIG. 1A) and a system (FIG. 1B) for predicting an outcome of a cardiac procedure in a subject.

FIG. 2 illustrates an exemplary risk prediction method that integrates biomarkers and risk factors extracted from multimodal data of echocardiogram, clinical parameters, and demographic information to achieve an accurate risk prevention.

FIGS. 3A and 3B illustrate receiver operating characteristics (ROC) curves of exemplary models for predicting the change of patients developing post-transplant lymphoproliferative disorder (PTLD) 3, 5, 10, and 15 years after heart transplantation. FIG. 3A shows the training results and FIG. 3B shows the testing results.

FIGS. 4A-4B illustrate an exemplary risk prediction model (FIG. 4A) and its design (FIG. 4B) using machine learning to predict post-operative risk.

FIG. 5 illustrates an exemplary risk prediction system with a front-end user interface and a backend server.

FIGS. 6A and 6B illustrate receiver operating characteristics (ROC) curves of exemplary models for predicting the change of patients developing PTLD. FIG. 6A shows the training results and FIG. 6B shows the testing results.

FIG. 7 shows the impact of multiple factors on model prediction accuracy, as quantified through Under the Receiver Operating Characteristic (AUROCs).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that include more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Heart failure (HF) places a growing clinical and economic burden in the United States. About 6.9 million Americans are affected by HF, identified on 379,800 death certificates (13.4%) in 2018. The annual incidence of HF is approximately 1 million. Despite the recent advances in pharmacotherapy and resynchronization, about 10% of HF patients have progressed to an advanced stage when conventional heart therapy and symptom management strategies are no longer working. These patients may be candidates for heart transplant or Left Ventricular Assist Device (LVAD) therapy. Despite the advancement of LVAD and heart transplant therapies, mortality and adverse events after the surgery remains high. Approximately 85 to 90 percent of heart transplant patients can live one year after their surgery, but the annual death rate of approximately 4 percent thereafter. About 33% of patients die or have poor quality of life over the year after the LVAD implantation. Therefore, it is important to perform risk assessment to determine which patients will benefit from these therapies.

There are a number of risk prediction scores at the present time, all of which have their advantages and limitations. The earliest of the models has been the Michigan right ventricular failure (RVF) risk score put forth in 2008, which was a single center retrospective study. Compared to other models, RVF was the most validated 16 times with a median c-statistic of 0.61. RVF used 4 binary pre-LVAD clinical variables. There was a higher concern for risk of bias due to variable RVF definition in the validation studies and indication bias due to inclusion of planned BIVAD patients and overfitting resulting in low RVF rates.

The EUROMACS model was similarly a retrospective study involving multiple centers using 5 binary variables for early RVF. The EUROMACS model was validated 5 times with a median c-statistic of 0.65. In the EUROMACS model, risk of bias was uncertain because the validation studies had variable definitions of RVF. Registry data was used which had the inherent problem of missing data. Moreover, the size of the cohort in the derivation study was large. Hence, the applicability concern was low.

The Penn model put forth in 2008 was a retrospective single center study for severe early RVF and used 6 binary variables. The study was validated 5 times with a median c-statistic of 0.63. Patients with planned biventricular assist devices (BIVAD) resulted in indication bias. RVF definitions varied, and the study was impacted by missing data and low RVF risk patients being excluded.

The Utah model was a single center, retrospective study with 8 categorical variables for early RVF. The model was validated 7 times with a median c-statistic of 0.55. However, problems with this model included variables that were overfitted and inadequately powered, patients with missing data, selection bias, and varied RVF definitions.

The CRITT model was also a single center, retrospective study that used central venous pressure of more than 15 mm Hg, severe RV dysfunction, preop mechanical ventilation and intubation, severe tricuspid regurgitation, and tachycardia for predicting risk for RVF. The model was validated 5 times with a median c-statistic of 0.63. The model was a single center retrospective study. This model had indication bias due to inclusion of planned BIVADs in the derivation study. Applicability was a concern due to a large number of pulsatile LVADs in the derivation batch and non-uniform RVF definitions in the validation batches.

Another model used 3 binary variables to predict early RVF. The model was validated 5 times with a median c-statistic of 0.61. This model was derived from a post hoc analysis of a cohort belonging to the multi-center HeartMate II trial. Hence, universal applicability was a concern. The model exhibited an inclusion bias because it only included a highly selected population who were all bridged to transplantation. This model had variable RVF definitions and lacked adequate power for analysis.

The Pittsburgh Decision Tree uses artificial intelligence (AI). The model was a single center, retrospective study which used 8 binary variables for early severe RVF. The model was validated 2 times with a median c-statistic of 0.53. Variable RVF definitions and low RVF rates and overfitting were all noted in this model.

Overall multiple limitations were noted in all of the existing risk models, thereby making them difficult to be universally applicable for RVF. The definitions for RVF used were highly varied. The percentage of continuous flow pumps were variable in the different studies, making them less predictable for the present day as pulsatile LVADs have essentially phased out.

Moreover, not all models reported calibration. Validation groups appear to have not been stringent in-patient selection or RVF definition, making them less reliable. Additionally, the type of RVF predicted whether acute, early, or late were highly varied. This led to heterogeneity, depending on the variability from institution to institution of medical versus device therapies for RVF. Existing studies have identified some risk factors for RVF prediction, but many clinically useful information from multimodal electronic health records (HER) data is overlooked.

Use of machine learning (ML) in developing risk scores for HF mortality seems to have an edge over conventional methods. For instance, The MARKER-HF score has a c-statistic of 0.88 and has been validated in 2 external study cohorts. This model used a boosted decision tree algorithm to derive a model based on automated training using two well defined cohorts—the low and high groups. In another study, telemetry data analyses from a wearable monitor used a general machine learning similarity-based modeling to predict HF hospitalization. Receiver operating characteristic curves showed a c-statistic of 0.86-89 using the analytics platform. The alert from such prediction models could help clinicians intervene before a HF hospitalization occurs. Prediction of mortality post LVAD implantation in general has been attempted using Bayesian network analysis with a c-statistic of 0.7 for 1-, 3- and 12-month mortality.

Applications of machine learning algorithms to assess tricuspid annulus excursion on 2-dimensional (2D) and 3-dimensional (3D) echocardiography have been attempted with considerable success in assessment of RV function. Application of an automated segmented model based on neural network architecture was used in a 2D echo image analysis. An ML algorithm was trained and tested in a 6-fold cross validation approach. Tricuspid annular displacement measurements using manual and automated ML segmentation showed that the automated approach was comparable to MRI data. The ROC curves used to test the model showed a c-statistic of 0.69-0.73 in a small population studied. The ML driven assessment used a deep learning framework and was time efficient with a processing time of less than 1 second. In another study, ML based algorithms using 3D echocardiographic images, RV volumes, and ejection fraction measurements were made with optimal reproducibility, suggesting that automated analysis of data may be more efficient.

A Bayesian network analysis driven model for acute, early, and late RVF post LVAD implantation was based on the INTERMACS registry. The acute, early, and late RVF models included 33, 34, and 33 preoperative variables (from demographics, hemodynamics, to laboratory values and medications) respectively. The performance of this model was superior to earlier models (c-statistic of 0.53-0.65) with c-statistics of 0.9, 0.83 and 0.88 for acute, early, and late RVF respectively. However, the study had limitations, such as missing data which are inherent to registry data.

In sum, ML has shown several successes in risk prediction for heart failure patients. However, available ML models for LVAD implantation and other cardiac procedures need additional validation. For instance, no ML models have been reported for heart transplant patients and no ML models have been reported for predicting risk of heart failure post cardiac surgery. The existing models are derived from retrospective analysis of pilot cohorts, and their prediction performance varies between health care centers and patient population. Numerous embodiments of the present disclosure aim to address the aforementioned limitations.

### Methods and Computing Devices for Predicting Cardiac Procedure Outcomes

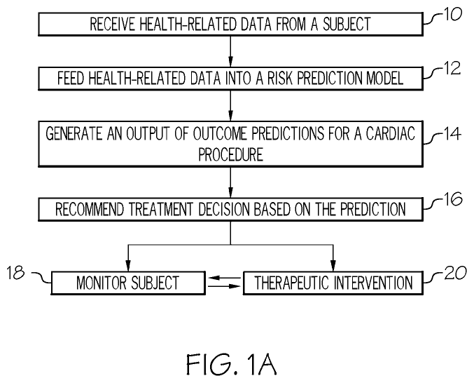

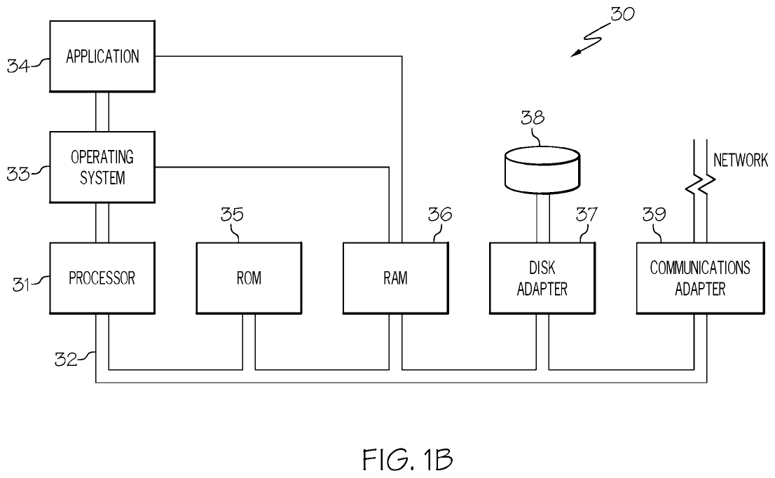

In some embodiments, the present disclosure pertains to a computer-implemented method of predicting an outcome of a cardiac procedure in a subject. In some embodiments illustrated in FIG. 1A, the methods of the present disclosure include receiving a plurality of health-related data from the subject (step 10); feeding the health-related data into a risk prediction model, which integrates the health-related data and predicts one or more outcomes of the cardiac procedure (step 12); and generating an output from the risk prediction model, where the output includes the predicted outcomes of the cardiac procedure (step 14). In some embodiments, the methods of the present disclosure also include a step of recommending a treatment decision based on the predicted outcomes of the cardiac procedure (step 16). In some embodiments, the treatment decision includes monitoring the subject (step 18) and/or administering a therapeutic intervention (step 20) to the subject.

Additional embodiments of the present disclosure pertain to a computing device that is operable for predicting an outcome of a cardiac procedure in a subject. In some embodiments, the computing device includes: (1) programming instructions for receiving a plurality of health-related data from the subject; (2) programming instructions for feeding the health-related data into a risk prediction model that is operable to integrate the health-related data and predict one or more outcomes of the cardiac procedure; and (3) programming instructions for generating an output from the risk prediction model, where the output includes the predicted outcomes of the cardiac procedure. In some embodiments, the computing devices of the present disclosure also include (4) programming instructions for recommending a treatment decision based on the predicted outcomes of the cardiac procedure.

As set forth in more detail herein, the methods and computing devices of the present disclosure can have numerous embodiments.

### Health-Related Data

The methods and computing devices of the present disclosure may utilize various types of health-related data. For instance, in some embodiments, the health-related data include, without limitation, data from a subject's echocardiogram, electronic health record (HER) data, health-related data in time series form, health-related data in time-invariant form, demographic information, or combinations thereof.

In some embodiments, the health-related data include two or more of heart ischemic time, creatinine levels, B2 antigen levels, DR1 antigen levels, DR15 antigen levels, DR2 antigen levels, DR51 antigen levels, HLA B antigen 38 levels, HLA B antigen 42 levels, bilirubin levels, gender assigned at birth, age, ethnicity, post graduate education status, blood type, diabetes status, cytomegalovirus infection status, immunosuppression status, immunosuppression status with steroids, immunosuppression status with azathioprine, immunosuppression status with anti-CD3 monoclonal antibodies, immunosuppression status with thymoglobulin, immunosuppression status with cyclosporine, Epstein Barr virus status, history of malignancy, coronary artery disease status, congenital heart defect status, presence of a ventricular assist device (VAD), anti-viral therapy status, or combinations thereof.

In some embodiments, the health-related data include Epstein Barr virus status, history of malignancy, immunosuppression status with cyclosporine, cytomegalovirus infection status, and post graduate education status. In some embodiments, the health-related data include Epstein Barr virus status, cytomegalovirus infection status, and post graduate education status. In some embodiments, the health-related data include Epstein Barr virus status, history of malignancy, immunosuppression status with steroids, blood type, and gender assigned at birth.

In some embodiments, the health-related data include Epstein Barr virus status, immunosuppression status with steroids, and ethnicity. In some embodiments, the health-related data include Epstein Barr virus status, history of malignancy, and gender assigned at birth. In some embodiments, the health-related data include Epstein Barr virus status, age, and immunosuppression status with anti-CD3 monoclonal antibodies. In some embodiments, the health-related data include Epstein Barr virus status, age, presence of a ventricular assist device (VAD), and anti-viral therapy status.

### Risk Prediction Models

The methods and computing devices of the present disclosure may utilize various types of risk prediction models. For instance, in some embodiments, the risk prediction model includes a machine-learning model trained on the plurality of health-related data. In some embodiments, the risk prediction model integrates the health-related data and predicts one or more outcomes of a cardiac procedure.

In some embodiments, the risk prediction model includes a random forest model. In some embodiments, the risk prediction model includes a gradient boosting model. In some embodiments, the gradient boosting model includes, without limitation, an adaptive boosting model, a random under sampling boosting model, or combinations thereof.

In some embodiments, the risk prediction model includes a regularized logistic regression model. In some embodiments, the regularized logistic regression model includes a Least Absolute Shrinkage and Selection Operator (LASSO) model. In some embodiments, the risk prediction model includes an encoder and a Dirichlet process mixture model (DPMM). In some embodiments, the risk prediction model adaptively clusters subjects (e.g., patients) into overlapping groups and estimates the data distribution using a combination of Neural Network-based survival kernel functions. In some embodiments, the encoder of the risk prediction model reduces the feature dimension to enable efficient Bayesian inference for large datasets.

The risk prediction models of the present disclosure may include various types of machine learning algorithms. For instance, in some embodiments, the machine-learning algorithm is an Li-regularized logistic regression algorithm. In some embodiments, the machine learning algorithm includes supervised learning algorithms. In some embodiments, the supervised learning algorithms include nearest neighbor algorithms, naïve-Bayes algorithms, decision tree algorithms, linear regression algorithms, support vector machines, neural networks, convolutional neural networks, ensembles (e.g., random forests and gradient-boosted decision trees), and combinations thereof.

The machine learning algorithms of the present disclosure may be trained in various manners. For instance, in some embodiments, the training includes (1) feeding a plurality of health-related data into a machine learning algorithm, where the health-related data are from one or more subjects that have or have not had certain cardiac procedures; (2) feeding another set of health-related data into the machine learning algorithm, where the health-related data are from one or more subjects that have or have not have or have not had certain cardiac procedures; and (3) training the machine learning algorithm to assess one or more predicted outcomes of a cardiac procedure by comparing the aforementioned categories of health-related data. In some embodiments, training a machine learning algorithm includes adjusting weights or parameters within the machine learning algorithm to differentiate between the aforementioned categories of health-related data. In some embodiments, training a machine learning algorithm includes providing health-related data relevance values to differentiate between the aforementioned categories.

### Predicted Outcomes of Cardiac Procedures

The methods and computing devices of the present disclosure may be utilized to predict the outcomes of various cardiac procedures. For instance, in some embodiments, the cardiac procedure includes heart transplantation. In some embodiments, the cardiac procedure includes Left Ventricular Assist Device (LVAD) implantation.

In some embodiments, the predicted outcome of a cardiac procedure includes one or more of risk of hospital re-admission, risk of mortality, risk of morbidity, risk of post-surgical complications, risk of heart failure, risk of cancer, risk of right ventricular failure, risk of right ventricular failure after Left Ventricular Assist Device (LVAD) implantation, or combinations thereof.

In some embodiments, the predicted outcome of a cardiac procedure includes a risk of cancer. In some embodiments, the cancer includes, without limitation, post-transplant lymphoproliferative disorder (PTLD), prostate cancer, lung cancer, and skin cancer.

In some embodiments, the predicted cancer includes post-transplant lymphoproliferative disorder (PTLD). In some embodiments, the predicted outcome includes risk of developing PTLD 1 year after the cardiac procedure. In some embodiments, the predicted outcome includes risk of developing PTLD 3 years after the cardiac procedure. In some embodiments, the predicted outcome includes risk of developing PTLD 5 years after the cardiac procedure. In some embodiments, the predicted outcome includes risk of developing PTLD 10 years after the cardiac procedure. In some embodiments, the predicted outcome includes risk of developing PTLD 15 years after the cardiac procedure.

In some embodiments, the predicted cancer includes skin cancer. In some embodiments, the predicted outcome includes risk of developing skin cancer 1 year after the cardiac procedure. In some embodiments, the predicted outcome includes risk of developing skin cancer 3 years after the cardiac procedure. In some embodiments, the predicted outcome includes risk of developing skin cancer 5 years after the cardiac procedure. In some embodiments, the predicted outcome includes risk of developing skin cancer 10 years after the cardiac procedure. In some embodiments, the predicted outcome includes risk of developing skin cancer 15 years after the cardiac procedure.

### Subjects

The methods and computing devices of the present disclosure may be utilized to predict the outcome of cardiac procedures in various subjects. For instance, in some embodiments, the subject is a human being. In some embodiments, the subject is expected to undergo a cardiac procedure. In some embodiments, the subject is a human being expected to undergo a cardiac procedure. In some embodiments, the expected cardiac procedure includes Left Ventricular Assist Device (LVAD) implantation. In some embodiments, the subject is suffering from heart failure. In some embodiments, the subject is suffering from heart failure and the expected cardiac procedure includes Left Ventricular Assist Device (LVAD) implantation.

### Treatment Decisions

In some embodiments, the methods of the present disclosure also include a step of recommending a treatment decision based on one or more predicted outcomes of the cardiac procedure. In some embodiments, the computing devices of the present disclosure also include programming instructions for recommending a treatment decision based on one or more predicted outcomes of the cardiac procedure.

In some embodiments, the methods of the present disclosure also include a step of implementing the recommended treatment decision. For instance, in some embodiments, a clinician or a healthcare provider may implement the recommended treatment decision.

The methods and computing devices of the present disclosure may be utilized to recommend various treatment decisions. For instance, in some embodiments, the treatment decision includes monitoring the subject for signs or symptoms of heart failure, cancer, cancer secondary to immunosuppression used to prevent transplant rejection, right ventricular failure, or combinations thereof. In some embodiments, the treatment decision includes administering a therapeutic intervention to the subject. In some embodiments, the therapeutic intervention includes, without limitation, a therapeutic agent to treat or prevent heart failure, a therapeutic agent to treat or prevent cancer, or combinations thereof.

### Generation of Outputs

The methods of the present disclosure may generate various outputs of predicted outcomes of cardiac procedures. Similarly, the computing devices of the present disclosure may include programming instructions for generating various outputs of predicted outcomes of cardiac procedures. For instance, in some embodiments, the output is generated in the form of a display on a digital screen. In some embodiments, the output is generated in the form of a printed report.

### Computing Device Architectures

The methods of the present disclosure can be implemented through the utilization of various computing devices with various architectures. Similarly, the computing devices of the present disclosure can include various architectures.

For instance, in some embodiments, the computing devices of the present disclosure are in electrical communication with a risk prediction model of the present disclosure. In some embodiments, the computing devices of the present disclosure include a web-based program, an application-based program, or combinations thereof. In some embodiments, the step of receiving health-related data includes entering the health-related data into the computing device. In some embodiments, programming instructions for receiving the health-related data include programming instructions for entering the health-related data into the computing device.

In some embodiments, the computing device includes a keyboard for a user to navigate and choose between different risk prediction functions. In some embodiments, the computing device further includes a display screen for displaying outputs from a risk prediction model.

The computing devices of the present disclosure can include various types of computer-readable storage mediums. In some embodiments, the computer-readable storage mediums can be a tangible device that can retain and store instructions for use by an instruction execution device. In some embodiments, the computer-readable storage medium may include, without limitation, an electronic storage device, a magnetic storage device, an optical storage device, an electromagnetic storage device, a semiconductor storage device, and combinations thereof. A non-exhaustive list of more specific examples of suitable computer-readable storage medium includes, without limitation, a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device, and combinations thereof.

A computer-readable storage medium, as used herein, is not to be construed as being transitory signals per se. Such transitory signals may be represented by radio waves or other freely propagating electromagnetic waves, electromagnetic waves propagating through a waveguide or other transmission media (e.g., light pulses passing through a fiber-optic cable), or electrical signals transmitted through a wire.

In some embodiments, computer-readable program instructions described herein can be downloaded to respective computing/processing devices from a computer-readable storage medium or to an external computer or external storage device via a network, such as the Internet, a local area network, a wide area network and/or a wireless network. In some embodiments, the network may include copper transmission cables, optical transmission fibers, wireless transmission, routers, firewalls, switches, gateway computers and/or edge servers. In some embodiments, a network adapter card or network interface in each computing/processing device receives computer-readable program instructions from the network and forwards the computer-readable program instructions for storage in a computer-readable storage medium within the respective computing/processing device.

In some embodiments, computer-readable program instructions for carrying out operations of the present disclosure may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object-oriented programming language such as Smalltalk, C++, or the like, and procedural programming languages, such as the “C” programming language or similar programming languages.

In some embodiments, the computer-readable program instructions may execute entirely on the user's computer as a stand-alone software package, partly on the user's computer and partly on a remote computer or entirely on the remote computer or server. In the latter scenario, the remote computer may be connected in some embodiments to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer-readable program instructions by utilizing state information of the computer-readable program instructions to personalize the electronic circuitry in order to perform aspects of the present disclosure.

Embodiments of the present disclosure as discussed herein may be implemented using a computing device illustrated in FIG. 1B. Referring now to FIG. 1B, FIG. 1B illustrates an embodiment of the present disclosure of the hardware configuration of a computing device 30 represents a hardware environment for practicing various embodiments of the present disclosure.

Computing device 30 has a processor 31 connected to various other components by system bus 32. An operating system 33 runs on processor 31 and provides control and coordinates the functions of the various components of FIG. 1B. An application 34 in accordance with the principles of the present disclosure runs in conjunction with operating system 33 and provides calls to operating system 33, where the calls implement the various functions or services to be performed by application 34. Application 34 may include, for example, a program for assessing a subject's predicted outcome for a cardiac procedure, such as in connection with FIGS. 1A, 2, 3A-3B, 4A-4B, 5, and 6A-6B.

Referring again to FIG. 1B, read-only memory (“ROM”) 35 is connected to system bus 32 and includes a basic input/output system (“BIOS”) that controls certain basic functions of computing device 30. Random access memory (“RAM”) 36 and disk adapter 37 are also connected to system bus 32. It should be noted that software components including operating system 33 and application 34 may be loaded into RAM 36, which may be computing device's 30 main memory for execution. Disk adapter 37 may be an integrated drive electronics (“IDE”) adapter that communicates with a disk unit 38 (e.g., a disk drive). It is noted that the program for assessing a subject's predicted outcome for a cardiac procedure, such as in connection with FIGS. 1A, 2, 3A-3B, 4A-4B, 5, and 6A-6B, may reside in disk unit 38 or in application 34.

Computing device 30 may further include a communications adapter 39 connected to bus 32. Communications adapter 39 interconnects bus 32 with an outside network (e.g., wide area network) to communicate with other devices.

Aspects of the present disclosure are described herein with reference to flowchart illustrations and/or block diagrams of methods, apparatus (systems), and computing devices according to embodiments of the disclosure. It will be understood that computer-readable program instructions can implement each block of the flowchart illustrations and/or block diagrams, and combinations of blocks in the flowchart illustrations and/or block diagrams.

These computer-readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer-readable program instructions may also be stored in a computer-readable storage medium that can direct a computer, a programmable data processing apparatus, and/or other devices to function in a particular manner, such that the computer-readable storage medium having instructions stored therein includes an article of manufacture including instructions which implement aspects of the function/act specified in the flowchart and/or block diagram block or blocks. The computer-readable program instructions may also be loaded onto a computer, other programmable data processing apparatus, or other devices to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computing devices according to various embodiments of the present disclosure. In this regard, each block in the flowchart or block diagrams may represent a module, segment, or portion of instructions, which includes one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

### ADDITIONAL EMBODIMENTS

Reference will now be made to more specific embodiments of the present disclosure and experimental results that provide support for such embodiments. However, Applicants note that the disclosure below is for illustrative purposes only and is not intended to limit the scope of the claimed subject matter in any way.

### Example 1. Systems for Predicting Treatment Outcomes and Adverse Events for Heart Failure Patients Undergoing Left Ventricular Assist Device (LVAD) Implantation or Heart Transplantation

This example discloses a system for predicting treatment outcomes and adverse events for heart failure patients undergoing Left Ventricular Assist Device (LVAD) implantation or heart transplantation. The technology includes novel components including, without limitation: use of new biomarkers and risk factors extracted from multimodal EHR data for risk prediction; new design of risk prediction models using machine learning methods for risk prediction; and design of a computing system with user interface to translate the risk prediction methods in clinical applications.

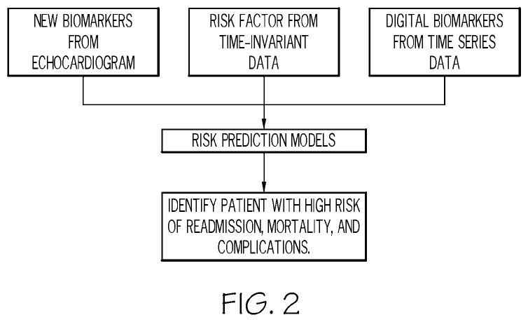

The risk prediction system disclosed leverages advanced data science and computing technologies, and multimodal electronic health records (HER) data of heart failure patients to accurately predict post-operative risk. FIG. 2 illustrates an exemplary risk prediction method that integrates biomarkers and risk factors extracted from multimodal data of echocardiogram, clinical parameters, and demographic information to achieve an accurate risk prevention.

The risk prediction method includes extracting biomarkers and risk factors from multimodal clinical (e.g., EHR) data, including biomarkers from echocardiograms, risk factors from time-invariant data, and digital biomarkers from time series data. Exemplary extracted biomarkers and risk factors greatly associated with post-transplant skin cancers include, for example, ischemic time, recipient serum creatinine, presences of B2, DR1, DR2 antigens in recipient serum, gender, diabetes in the recipient, ethnicity, and CMV (cytomegalovirus) status of the transplant donor and induction with azathioprine, OKT3, and thymoglobulin or cyclosporine. Biomarkers and risk factors highly associated with PTLD development 5 years after transplantation include positive Epstein Barr virus status, history of malignancy, induction with cyclosporine, coronary artery disease, negative DR51 in the donor, donor's HLA DR antigen 15, congenital heart defect, male gender, donor's HLA B antigen 38, and positive donor Epstein Barr IgG. Additionally, biomarkers and risk factors for 3-year PTLD development include positive Epstein Barr virus status, history of malignancy, induction with cyclosporine, CMV match, and postgraduate education. Furthermore, extracted biomarkers and risk factors for 1-year PTLD development include positive Epstein Barr virus status, CMV match, post graduate education, anti-rejection with steroids, and induction with steroids. Moreover, black race was identified as having a protective effect against post-transplant PTLD.

The risk prediction system uses an unsupervised image analysis tool combining a recurrent neural network, a convolutional neural network and a transformer to extract new digital biomarkers and risk factors from echocardiograms. The biomarkers and risk factors are identified as highly correlated with post-operative mortality and complications such as right ventricular failure after LVAD implantation.

The extracted biomarkers and risk factors are integrated into a risk prediction model that outputs identification of patients that are more likely to be readmitted, develop complications and/or permanent disability.

The risk prediction method includes Least Absolute Shrinkage and Selection Operator (LASSO) Logistic Regression models that predict post-transplant lymphoproliferative disorder (PTLD) 1-year, 3-years, and 5-years following heart transplantation. The models have mean±standard deviation Area Under the Receiver Operating Characteristic (AUROCs) of 0.81±0.036 (1 year), 0.73±0.025 (3-years), 0.70±0.019 (5 years) on the training data and 0.714±0.052 (1-year), 0.67±0.043 (3-years), and 0.66±0.023 (5-years) on the testing data. Important risk factors of PTLD 1 year after transplant include negative Epstein-Barr virus, malignancy history, immunosuppression with steroids, donor blood type O, and male gender, 3 years after transplant are negative Epstein-Barr virus and immunosuppression with steroids, and 5 years after heart transplant are negative Epstein-Barr virus, malignancy history, positive donor Epstein-Barr IgG, and male gender.

The risk prediction method combines multiple machine learning models to predict cancers (for example, prostate, lung, and skin cancers, and PTLD) following heart transplantation. These models consist of Random Forest and boosting models such as Gradient Boosting, Adaptive Boosting, and Random Under sampling Boosting.

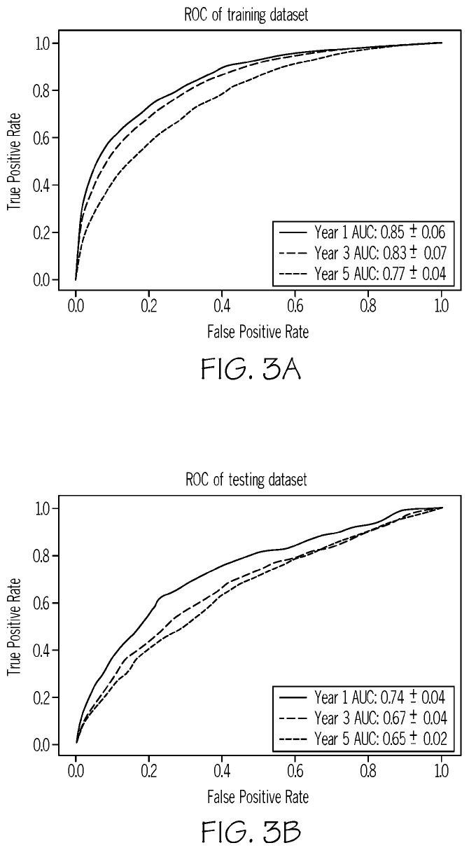

FIGS. 3A-3B illustrate receiver operating characteristics (ROC) curves of exemplary models for predicting the chance of patients developing PTLD 1, 3, 5 years after heart transplantation. FIG. 3A shows the training results and FIG. 3B shows the testing results. The model has AUROCs±standard deviation of 0.851±0.071, 0.831±0.073, 0.772±0.049 1, 3, and 5 years after transplantation respectively in the training data and 0.735±0.048, 0.669±0.048, 0.649±0.027 1, 3, 5 years after transplantation respectively in the testing data.

The most important variable for 1-year prediction is Epstein-Barr virus, for 3-year prediction are Epstein-Barr virus, age, and immunosuppression using OKT3, for the 5-year prediction are Epstein-Barr virus, age, ventricular assist device (VAD) indicator, and anti-viral therapy.

The risk prediction system includes a deep neural network-based survival model and a novel learning framework built upon an adaptive clustering strategy and weighted maximum likelihood estimation to address data imbalance issues and account for patient heterogeneity. The models achieve mean AUROCs of 0.851, 0.842, 0.833, and 0.831 on the training data and mean AUROCs of 0.824, 0.807, 0.808, and 0.817 on the testing data for predicting skin cancer at 3, 5, 10, and 15 years after heart transplantation. The concordance values for the training and testing data are 0.832 and 0.756, respectively.

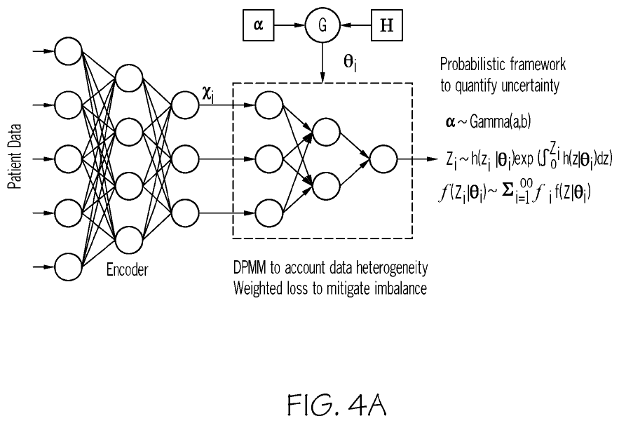

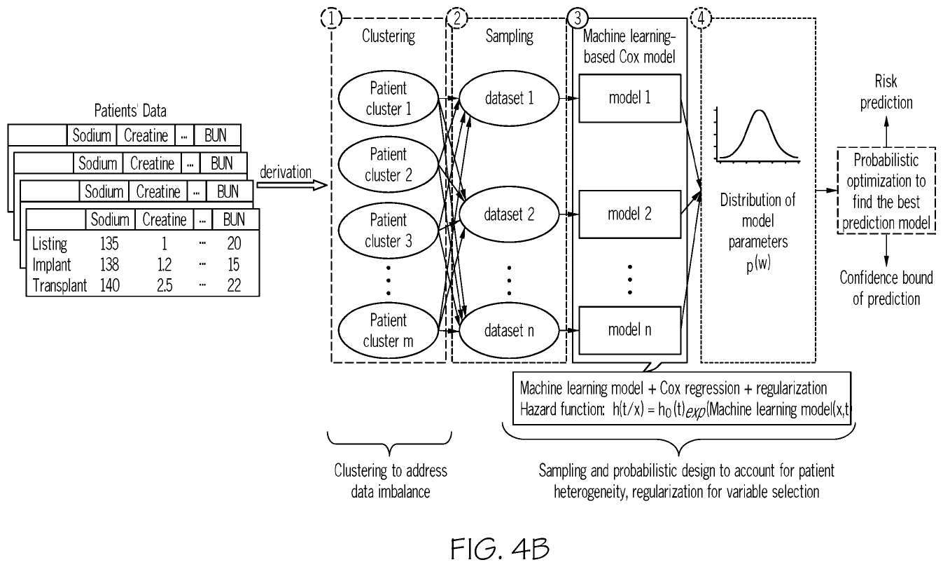

FIGS. 4A-4B illustrates an exemplary risk prediction model that utilizes machine learning to forecast post-operative risks. The model incorporates an autoencoder along with a Dirichlet process mixture survival model to predict adverse events. It tackles various modeling challenges, including the quantification of uncertainty, handling heterogeneity, and addressing data imbalance issues.

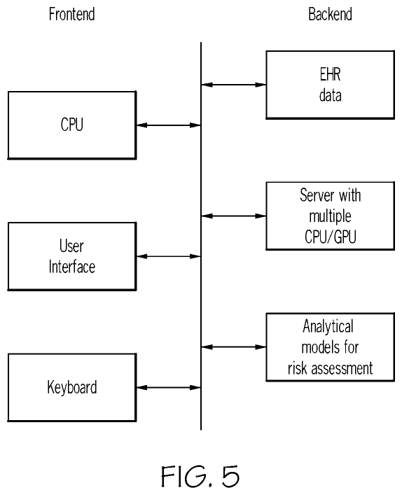

FIG. 5 illustrates an exemplary risk prediction system with a front-end user interface and a backend server. The front end includes a central processing unit (CPU), a web-based user interface to display risk prediction results, and a keyboard for user to navigate and choose between different risk prediction functions. The backend includes a multi-core CPU/GPU server, the risk prediction models, and connection to patients' database.

### Example 2. LASSO Logistic Regression Models to Predict Post-Transplant Lymphoproliferative Disorder in Heart Transplant Recipients

Post-Transplant Lymphoproliferative Disorder (PTLD) is a fatal malignancy happening in heart transplant recipients. This Example attempted to identify risk factors and generate a regularized logistic regression model for predicting risk of PTLD at 1-, 3- and 5-years post heart transplant using the Scientific Registry of Transplant Recipients (SRTR) database.

The models considered heart transplant recipients from 1987 to 2021 contained in the SRTR. After excluding retransplants, patients with multiple organ transplants and patients aged younger than 18, 55150 patients were identified out of which 1742 patients had PTLD. Least Absolute Shrinkage and Selection Operator (LASSO) Logistic Regression models were developed to predict PTLD 1-year, 3-years, and 5-years following transplant. 5-fold cross validation was performed to train, tune, and test the model performance. The data was split 65% for training, 15% for tuning the regularization parameter, and 20% for testing. The discrimination performance was assessed using the mean Area Under the Receiver Operating Characteristic Curve (AUROC).

Important variables were identified by looking at the largest non-zero coefficients of the LASSO models. Odds Ratio (OR) were computed using non-regularized logistic regression models for 1-year, 3-years and 5-years including the variables with non-zero coefficients from the LASSO models for the same time periods. The Scikit-learn library from Python was used to develop the LASSO models and ORs were computed using the R statistical programming language.

A total of 84 variables were extracted from the SRTR and included in the LASSO Logistic Regression models. The models had mean±std AUROCs of 0.81±0.036 (1 year), 0.73±0.025 (3-years), 0.70±0.019 (5 years) on the training data and 0.714±0.052 (1-year), 0.67±0.043 (3-years), and 0.660±0.023 (5-years) on the test data. Important variables identified from 1-year model are: positive Epstein-Barr virus (OR, 0.087; PC0.01), malignancy history (OR, 3.1; PC0.01), immunosuppression with steroids (OR, 1.85; P=0.01), donor blood type O (OR, 1.70; P=0.096; reference category=donor blood type A) and male gender (OR, 1.57; P=0.066). Important variables from 3-years model are: positive Epstein-Barr virus (OR, 0.22; P<0.01), black race (OR, 0.40; P=0.02), and immunosuppression with steroids (OR, 1.31; P=0.08). Important variables from 5-years model are: positive Epstein-Barr virus (OR, 0.27; P<0.01), malignancy history (OR, 2.3; P<0.01), positive donor Epstein-Barr IgG (OR, 5.0, P=0.02), and male gender (OR, 1.4; P=0.009).

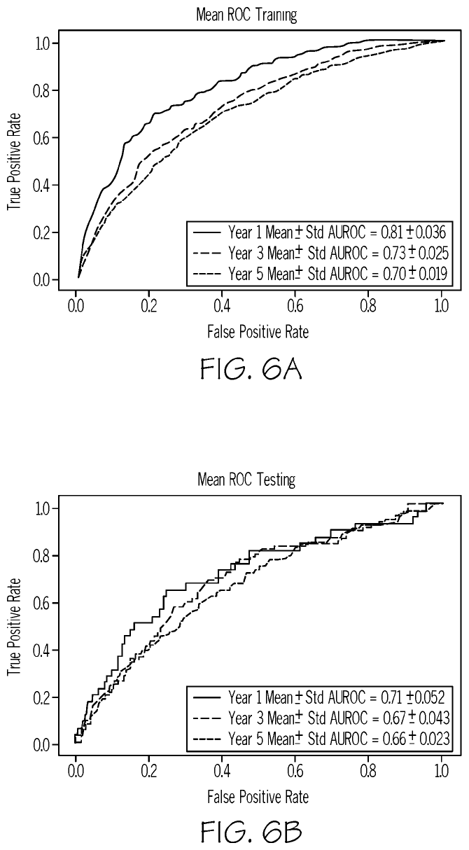

FIGS. 6A-6B illustrate receiver operating characteristics (ROC) curves of exemplary models for predicting the chance of patients developing PTLD 1, 3, 5 years after heart transplantation. FIG. 6A shows the training results and FIG. 6B shows the testing results.

The mean AUROC metric shows that the 1-year model possesses better discriminatory capacity than the 3-years and 5-years models. This Example is limited by its retrospective nature and internal validation.

### Example 3. Machine Learning Ensemble Models for Predicting PTLD in Heart Transplant Recipients

This Example aimed to develop machine learning models based on bagging and boosting ensembles of trees. The objective is to predict PTLD at 1-, 3- and 5-years post heart transplant using the Scientific Registry of Transplant Recipients (SRTR).

Heart transplant recipients from 1987 to 2021 were extracted from the SRTR. After excluding retransplants, patients with multiple organ transplants and patients aged younger than 18, 55,150 patients were identified out of which 1,742 patients had PTLD. As summarized in Table 1, machine learning models were developed to predict PTLD 1 year, 3 years, and 5 years following the transplant. The models consist of Random Forest and three boosting models (Gradient Boosting, Adaptive Boosting, and Random Undersampling Boosting). 5-fold cross validation was performed to train, tune, and test the models. The data was split 65% for training, 15% for tuning model hyperparameters, and 20% for testing. The discrimination performance was assessed using the Area Under the Receiver Operating Characteristic Curve (AUROC). Permutation-based Variable Importance (VIMP) was performed to assess the importance of each variable by degradation in the model's discrimination performance (decrease in AUROC). The Scikit-learn library from Python was used for all analysis.

A total of 84 variables were extracted from the SRTR and included in the models. The Gradient Boosting model has the best training and testing AUROCs compared to the rest models. Its AUROCs are 0.851±0.071, 0.831±0.073, 0.772±0.049 1, 3, and 5 years after transplantation, respectively in the training data and 0.735±0.048, 0.669±0.048, 0.649±0.027 1, 3, 5 years after transplantation respectively in the testing data. The most important variable for the 1-year Gradient Boosting model is Epstein-Barr virus with a 9.3% degradation in AUROC. Most important variables for the 3-year Gradient Boosting model are Epstein-Barr virus (2.5% decrease in AUROC), age (1.3% decrease in AUROC) and immunosuppression using OKT3 (0.66% decrease in AUROC). Most important variables for the 5-year Gradient Boosting model are Epstein-Barr virus (1.58% decrease in AUROC), age (0.77% decrease in AUROC), ventricular assist device (VAD) indicator (0.55% decrease in AUROC) and anti-viral therapy (0.50% decrease in AUROC).

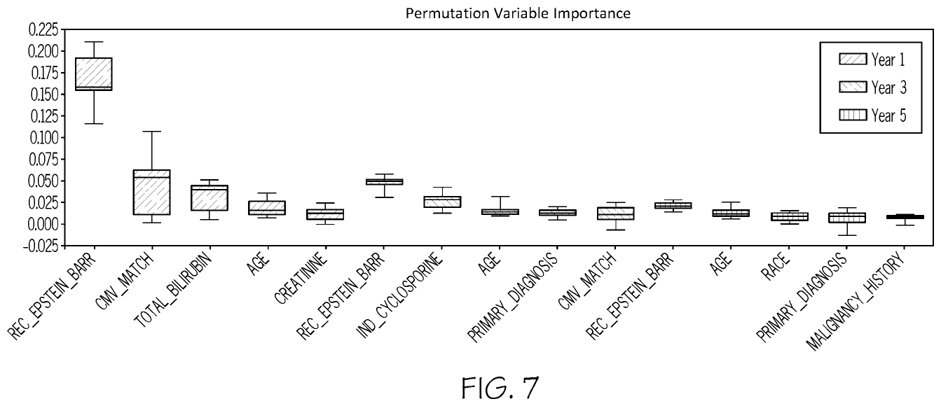

In sum, the Gradient Boosting model possesses the best discrimination capacity. However, it also has a higher standard deviation, indicating that it tends to overfit more than the other ensemble models. 1-year models possess better discrimination than 3-years and 5-year models. Epstein-Barr virus and age are important for 1-, 3-, and 5-year predictions of PTLD after heart transplantation. CMV match, total bilirubin, and creatinine contribute to the 1-year prediction; CMV match, induction with cyclosporine, and primary diagnosis contribute to the 3-year prediction; and race, primary diagnosis, and malignancy history contribute to the 5 year prediction. FIG. 7 shows the impact of these factors on the model prediction accuracy quantified through AUROCs. The study is limited by its retrospective nature and internal validation.

TABLE 1
Machine learning models developed to predict PTLD 1 year, 3 years, and 5 years following heart transplant.

| | Training AUROC (Mean ± Std) | | | Testing AUROC (Mean ± Std) | | |
|---|---|---|---|---|---|---|
| Model | 1-year | 3-years | 5-years | 1-year | 3-years | 5-years |
| Random Forest | 0.814 ± 0.039 | 0.694 ± 0.036 | 0.673 ± 0.018 | 0.706 ± 0.041 | 0.648 ± 0.046 | 0.644 ± 0.024 |
| Gradient Boosting | 0.851 ± 0.071 | 0.831 ± 0.073 | 0.772 ± 0.049 | 0.735 ± 0.048 | 0.669 ± 0.048 | 0.649 ± 0.027 |
| AdaBoost | 0.811 ± 0.028 | 0.719 ± 0.006 | 0.693 ± 0.035 | 0.685 ± 0.015 | 0.664 ± 0.042 | 0.646 ± 0.015 |
| RUSBoost | 0.813 ± 0.033 | 0.748 ± 0.031 | 0.706 ± 0.037 | 0.717 ± 0.036 | 0.65 ± 0.035 | 0.646 ± 0.015 |

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A computer-implemented method of predicting an outcome of a cardiac procedure in a subject, said method comprising:
receiving a plurality of health-related data from the subject;
feeding said health-related data into a risk prediction model, wherein the risk prediction model comprises a machine-learning model trained on the plurality of health-related data, and wherein the risk prediction model integrates the health-related data and predicts one or more outcomes of the cardiac procedure; and
generating an output from the risk prediction model, wherein the output comprises the one or more predicted outcomes of the cardiac procedure.

2. The method of claim 1, wherein the health-related data are selected from the group consisting of data from the subject's echocardiogram, electronic health record (EHR) data, health-related data in time series form, health-related data in time-invariant form, demographic information, or combinations thereof.

3. The method of claim 1, wherein the health-related data comprise two or more of heart ischemic time, creatinine levels, B2 antigen levels, DR1 antigen levels, DR15 antigen levels, DR2 antigen levels, DR51 antigen levels, HLA B antigen 38 levels, HLA B antigen 42 levels, bilirubin levels, gender assigned at birth, age, ethnicity, post graduate education status, blood type, diabetes status, cytomegalovirus infection status, immunosuppression status, immunosuppression status with steroids, immunosuppression status with azathioprine, immunosuppression status with anti-CD3 monoclonal antibodies, immunosuppression status with thymoglobulin, immunosuppression status with cyclosporine, Epstein Barr virus status, history of malignancy, coronary artery disease status, congenital heart defect status, presence of a ventricular assist device (VAD), anti-viral therapy status, or combinations thereof.

4. The method of claim 1, wherein the predicted outcome of the cardiac procedure comprises one or more of risk of hospital re-admission, risk of mortality, risk of morbidity, risk of post-surgical complications, risk of heart failure, risk of cancer, risk of right ventricular failure, risk of right ventricular failure after Left Ventricular Assist Device (LVAD) implantation, or combinations thereof.

5. The method of claim 1, wherein the predicted outcome of the cardiac procedure comprises a risk of cancer.

6. The method of claim 5, wherein the cancer is selected from the group consisting of post-transplant lymphoproliferative disorder (PTLD), prostate cancer, lung cancer, and skin cancer.

7. The method of claim 5, wherein the cancer comprises skin cancer.

8. (canceled)

9. The method of claim 1, wherein the cardiac procedure comprises heart transplantation, and wherein the predicted outcome of the cardiac procedure comprises a risk of skin cancer.

10. (canceled)

11. The method of claim 1, wherein the subject is a human being in need of heart transplantation.

12. (canceled)

13. The method of claim 1, wherein the risk prediction model comprises a random forest model, a gradient boosting model, a regularized logistic regression model, a regularized logistic regression model comprising a Least Absolute Shrinkage and Selection Operator (LASSO) model, an encoder and a Dirichlet process mixture model (DPMM), or combinations thereof.

14. (canceled)

15. The method of claim 1, wherein the risk prediction model comprises a gradient boosting model selected from the group consisting of an adaptive boosting model, a random under sampling boosting model, or combinations thereof.

16-18. (canceled)

19. The method of claim 1, further comprising a step of recommending a treatment decision based on the one or more predicted outcomes of the cardiac procedure.

20. The method of claim 19, wherein the treatment decision comprises monitoring the subject for signs or symptoms of heart failure, cancer, right ventricular failure, or combinations thereof.

21. The method of claim 19, wherein the treatment decision comprises administering a therapeutic intervention to the subject.

22. The method of claim 21, wherein the therapeutic intervention is selected from the group consisting of a therapeutic agent to treat or prevent heart failure, a therapeutic agent to treat or prevent cancer, or combinations thereof.

23. The method of claim 19, further comprising a step of implementing the recommended treatment decision.

24. The method of claim 1, wherein the method occurs through the utilization of an application-based program.

25. A computing device operable for predicting an outcome of a cardiac procedure in a subject, said computing device comprising:
programming instructions for receiving a plurality of health-related data from the subject;
programming instructions for feeding said health-related data into a risk prediction model, wherein the risk prediction model comprises a machine-learning model trained on the plurality of health-related data, and wherein the risk prediction model is operable to integrate the health-related data and predict one or more outcomes of the cardiac procedure; and
programming instructions for generating an output from the risk prediction model, wherein the output comprises the one or more predicted outcomes of the cardiac procedure.

26. The computing device of claim 25, wherein the health-related data are selected from the group consisting of data from the subject's echocardiogram, electronic health record (EHR) data, health-related data in time series form, health-related data in time-invariant form, demographic information, or combinations thereof.

27. The computing device of claim 25, wherein the health-related data comprise two or more of heart ischemic time, creatinine levels, B2 antigen levels, DR1 antigen levels, DR15 antigen levels, DR2 antigen levels, DR51 antigen levels, HLA B antigen 38 levels, HLA B antigen 42 levels, bilirubin levels, gender assigned at birth, age, ethnicity, post graduate education status, blood type, diabetes status, cytomegalovirus infection status, immunosuppression status, immunosuppression status with steroids, immunosuppression status with azathioprine, immunosuppression status with anti-CD3 monoclonal antibodies, immunosuppression status with thymoglobulin, immunosuppression status with cyclosporine, Epstein Barr virus status, history of malignancy, coronary artery disease status, congenital heart defect status, presence of a ventricular assist device (VAD), anti-viral therapy status, or combinations thereof.

28. The computing device of claim 25, wherein the predicted outcome of the cardiac procedure comprises one or more of risk of hospital re-admission, risk of mortality, risk of morbidity, risk of post-surgical complications, risk of heart failure, risk of cancer, risk of right ventricular failure, risk of right ventricular failure after Left Ventricular Assist Device (LVAD) implantation, or combinations thereof.

29. The computing device of claim 25, wherein the predicted outcome of the cardiac procedure comprises a risk of cancer.

30. The computing device of claim 29, wherein the cancer is selected from the group consisting of post-transplant lymphoproliferative disorder (PTLD), prostate cancer, lung cancer, and skin cancer.

31. The computing device of claim 29, wherein the cancer comprises skin cancer.

32-33. (canceled)

34. The computing device of claim 25, wherein the subject is a human being in need of heart transplantation, and wherein the cardiac procedure comprises heart transplantation.

35-36. (canceled)

37. The computing device of claim 25, wherein the risk prediction model comprises a random forest model, a gradient boosting model, a regularized logistic regression model, a regularized logistic regression model comprising a Least Absolute Shrinkage and Selection Operator (LASSO) model, an encoder and a Dirichlet process mixture model (DPMM), or combinations thereof.

38. (canceled)

39. The computing device of claim 25, wherein the risk prediction model comprises a gradient boosting model selected from the group consisting of an adaptive boosting model, a random under sampling boosting model, or combinations thereof.

40-42. (canceled)

43. The computing device of claim 25, wherein the computing device further comprises programming instructions for recommending a treatment decision based on the one or more predicted outcomes of the cardiac procedure.

44. The computing device of claim 43, wherein the treatment decision comprises monitoring the subject for signs or symptoms of heart failure, cancer, right ventricular failure, or combinations thereof.

45. The computing device of claim 43, wherein the treatment decision comprises administering a therapeutic intervention to the subject.

46. The computing device of claim 25, wherein the therapeutic intervention is selected from the group consisting of a therapeutic agent to treat or prevent heart failure, a therapeutic agent to treat or prevent cancer, or combinations thereof.

47. The computing device of claim 25, wherein the computing device comprises an application-based program.

48. The method of claim 1, wherein the risk prediction model adaptively clusters subjects into groups.

49. The computing device of claim 25, wherein the risk prediction model adaptively clusters subjects into groups.
